(19) United States
(12) Patent Application Publication
(10) Pub. No.: US 2005/0266399 A1
(43) Pub. Date: Dec. 1, 2005

(54) HCV regulated protein expression

(76) Inventors: Peter Berndt, Basel (CH); Peter Michael Kilby, Wokingham (GB); Paul Rugman, Quorn (GB)

Correspondence Address: HOFFMANN-LA ROCHE INC.;PATENT LAW DEPARTMENT, 340 KINGSLAND STREET, NUTLEY, NJ, 07110, US

(21) Appl. No.: 10/878,556

(22) Filed: Jun. 28, 2004

(30) Foreign Application Priority Data
Jun. 30, 2003 (GB) 0315248.5

(51) Int. Cl. C12Q 1/70

(52) U.S. Cl. 435/5

(57) ABSTRACT

The present invention relates to novel host cell targets for antiviral, preferably anti-hepatitis C virus (HCV), compounds identified by analysis of HCV replicon-regulated polypeptide expression, to prognostic markers for the prediction of the outcome of a viral infection, to in vitro methods for the prediction of the outcome of a HCV infection in a subject, to screening methods for identifying compounds which interact with and/or modulate the activity of the novel host cell targets or which modulate the expression of said novel host cell targets.

### BACKGROUND OF THE INVENTION

Hepatitis C virus (HCV) infection is a rapidly increasing public health problem, with an estimated 200 million chronically infected patients worldwide. No vaccines are currently available (Hagedom, C. H. & Rice, C. M. eds. (1999) Current Topics in Microbiology and Immunology (Springer, Berlin)) for HCV, and only a subset of patients responds to current treatments. The mechanisms of viral infection, persistence and clearance are not well understood, mainly because of lack of adequate animal models and cell culture systems.

As the propagation of the virus in cell culture has been unsuccessful (Tan, S. -L. et al. (2002), Hepatitis C Therapeutics: Current Status and Emerging Strategies, Nature Reviews, Vol. 1, 867-881), a HCV-replicon system, in which expression of the HCV non-structural proteins drives the replication of a subgenomic HCV RNA (Bartenschläger, R. (2002), Hepatitis C virus replicons: potential role for drug development, Nature Reviews, Vol. 1, 911-916) has been generated. With this system in hand, HCV replication and host-all response can be studied in greater detail. Although most of the current antiviral efforts are aimed at key viral enzymes that are essential to HCV replication, cellular genes that are required for HCV replication represent an attractive class of genes for targeting to control viral replication, e.g., with small molecules.

mRNA viruses are totally dependent on the host cell for protein synthesis. They have to subvert the host cell protein synthesis machinery to a significant degree in order to express their viral proteins. Not surprisingly, parts of this process are already described in the art. Thus, Krüger et al. (Krüger, M. et al., Identification of eIF2Bγ and eIF2γ as cofactors of HCV IRES-mediated translation using a functional genomics approach, PNAS, 2000, vol. 97, 8566-8571) used a functional genomics approach to identify eIF2Bγ and eIF2γ cellular cofactors involved in HCV IRES function.

During viral assembly, mammalian viruses are dependent on the host-cell glycosylation machinery for morphogenesis and secretion. The first step in the N-linked glycosylation pathway is accomplished by the ER-localized glucosidases, which may be targeted at a low level to treat viral infections without compromising the host. Su A. J. (Su A I, Pezacki J P et al, PNAS, 2002, 99(24), 15669-15674) showed that among the host genes induced in HCV infection were several involved in the immune response and induced by IFN-γ as CD8, MHC Class II components, chemokines as RANTES and MIG. Other genes identified that were differentially induced or repressed during early HCV infection were associated with lipid metabolism (prognostic markers of outcome).

However, it is well known that regulation on the transcriptional level is not necessarily indicative of a similar regulation of the expression of the respective gene on the translational level. Several studies have shown that mRNA expression levels and actual protein expression are only poorly correlated (Gygi S P, Rochon Y, Franza B R, Aebersold R., Mol Cell Biol., 1999, 19(3), 1720-30).

Therefore, there is a need in the art for novel host cell targets in the treatment of a HCV infection, as well as for markers to study and to predict the outcome of a HCV infection.

### SUMMARY OF THE INVENTION

The present invention relates to novel host cell targets for antiviral compounds, preferably anti-HCV compounds, identified by analysis of HCV replicon-regulated polypeptide expression, to prognostic markers for the prediction of the outcome of a viral infection, to in vitro methods for the prediction of the outcome of a HCV infection in a subject, to screening methods for identifying compounds which interact with and/or modulate the activity of the novel host cell targets or which modulate the expression of said novel host cell targets. The polypeptides as listed in Tables 1 and 2 are down- or up-regulated by the presence of HCV replicon-derived RNA and it is accordingly possible to use them as novel targets for viral drugs, and as prognostic markers for the prediction of the disease outcome.

### DETAILED DESCRIPTION OF THE INVENTION

The present invention provides a target for an antiviral compound comprising at least one polypeptide selected from the group of polypeptides as listed in Tables 1 and 2. Preferably, a target for an antiviral compound comprising at least one polypeptide selected from the group of polypeptides as listed in Table 3 is provided. The polypeptides as listed in Table 3 comprise up- or down-regulated mRNA chaperones, mRNA-binding proteins such as, e.g., PTB. Also preferably, a target for an antiviral compound comprising at least one polypeptide selected from the group of polypeptides as listed in Table 4 is provided. The polypeptides as listed in Table 4 comprise up- or down-regulated translation initiation proteins such as, e.g., eIF2γ and eIF2Bγ. In further preferred embodiment, a target for an antiviral compound comprising at least one polypeptide selected from the group of polypeptides as listed in Table 5 is provided. The polypeptides as listed in Table 5 comprise up- or down-regulated polypeptides involved in lipid metabolism, e.g., polypeptides involved in cholesterol synthesis and β-oxidation. In another preferred embodiment, a target for an antiviral compound comprising at least one polypeptide selected from the group of polypeptides as listed in Table 6 is provided. The polypeptides as listed in Table 6 comprise up- or down-regulated polypeptides involved in signal transduction pathways, e.g., MAP kinases as MK01 or oncogenes as SRC8. Preferred are the above-described targets as anti-HCV targets. The target for an antiviral compound, preferably an anti-HCV compound may comprise two, three, four, five, six or even more of the polypeptides as listed in Table 1, Table 2, preferably in Table 3, in Table 4, in Table 5, and in Table 6. Especially preferred as targets for an anti-HCV compound are the polypeptides of SEQ ID NO: 70 (PTB_HUMAN), 101 (NR54_HUMAN), 72 (ACDB_HUMAN), 193 (CHR1-O14805), or 166 (SCR8_HUMAN). The antiviral compound as used herein may comprise compounds against any virus e.g. DNA and RNA virus, preferably they comprise compounds against RNA viruses e.g. HIV, poliovirus. Most preferably, the antiviral compounds are compounds against HCV.

The term “polypeptide” as used herein, refers to a polymer of amino acids, and not to a specific length. Thus, peptides, oligopeptides and proteins are included within the definition of polypeptide.

The present invention also provides a prognostic marker for the prediction of the outcome of a viral infection comprising at least one polypeptide selected from group of polypeptides as listed in Tables 1 and 2. Preferably, a prognostic marker for the prediction of the outcome of a viral infection comprising at least one polypeptide selected from the group of polypeptides as listed in Tables 3 to 6 is provided. The marker may comprise two, three, four, five six, seven, eight, nine, ten or more of the polypeptides selected from the polypeptides as listed in Tables 1 and 2 or as listed in Tables 3 to 6. Preferably, the invention provides a prognostic marker for the prediction of the outcome of a HCV infection. The outcome of a HCV infection may comprise hepatic failure, hepatocellular carcinoma and liver cirrhosis (Hofnagle, J. H., Hepatitis C—the clinical spectrum of disease Hepatology 26, 1997, 15-20).

The present invention also provides an in vitro method for the prediction of the outcome of a HCV infection in a subject comprising the steps of obtaining a biological sample from a HCV-infected subject; and determining the expression level of at least one polypeptide from the group of polypeptides as listed in Tables 1 to 6. The term “determining the expression level” as used herein refers to the quantitative determination of the expression of polypeptides in biological samples from an HCV-infected subject. Moreover, the in vitro method further relates to comparing the expression level of the at least one polypeptides from the group of polypeptides as listed in Tables 1 to 6 to its expression level from different time points in the course of the HCV infection and/or to the differences in expression to biological samples from uninfected subjects. Methods for determining the expression level of polypeptides in biological samples are well known in the art and include, but are not limited to, Western-blotting, ELISA or RIA. Antibodies recognizing the polypeptides listed in Tables 1 to 6 can either be generated for the purpose of detecting said polypeptides, e.g., by immunizing rabbits with purified proteins, or known antibodies recognizing said polypeptides can be used. For example, an antibody capable of binding to the denatured proteins, such as a polyclonal antibody, can be used to detect the polypeptides of this invention in a Western Blot. An example for such a method to determine the expression level of a prognostic marker is an ELISA. This type of protein quantification is based on an antibody capable of capturing a specific antigen, and a second antibody capable of detecting the captured antigen. A further method for the determination of the expression of a marker for the prediction of the outcome of a HCV infection comprises analysing biopsy specimens for the presence or absence of the markers of this invention. Methods for the detection of these markers are well known in the art and include, but are not limited to, immunohistochemistry or immunofluorescent detection of the presence or absence of the polypeptides of this invention. Methods for preparation and use of antibodies, and the assays mentioned hereinbefore are described in Harlow, E. and Lane, D. Antibodies: A Laboratory Manual, (1988), Cold Spring Harbor Laboratory Press.

For the methods for the prediction of the outcome of a HCV infection of the present invention, suitable biological samples need to be analysed for the expression of a marker. Said biological samples can be serum, plasma, or liver tissue. Cells from liver tissue can be obtained by, e.g., large-bore needle biopsy.

In the present invention, the term subject comprises any mammal, which may be infected with HCV. Preferably, the subject is a human being.

The present invention also provides a screening method for identifying and/or obtaining a compound which interacts with a polypeptide selected from the polypeptides as listed in Tables 1 to 6 whose expression is up-regulated or down-regulated in HCV-infected tissue comprising the steps of contacting said polypeptide with a compound or a plurality of compounds under conditions which allow interaction of said compound with said polypeptide; and by detecting the interaction between said compound or plurality of compounds with said polypeptide.

The “interaction” in the screening methods as disclosed herein may be measured by conventional methods. The type of conventional method for testing the interaction of a compound with a polypeptide that is soluble, as opposed to membrane associated, can be an in vitro method using either purified recombinant polypeptide, or native polypeptide purified from cells that endogenously express the polypeptide. As a non-limiting example, a polypeptide of the invention can be bound to beads or immobilized on plastic or other surfaces, and interaction of a compound with the polypeptide can be measured by either using a labeled compound and measuring the label bound to the polypeptide, or by displacement of a labeled known ligand from said polypeptide.

For polypeptides that are associated with the cell membrane on the cell surface, or which are expressed as transmembrane or integral membrane polypeptides, the interaction of a compound with said polypeptides can be detected with different methods which include, but are not limited to, methods using cells that either normally express the polypeptide or in which the polypeptide is overexpressed, e.g., by detecting displacement of a known ligand which is labeled by the compound to be screened. Alternatively, membrane preparations may be used to test for interaction of a compound with such a polypeptide.

Interaction assays to be employed in the method disclosed herein may comprise FRET-assays (fluorescence resonance energy transfer; as described, inter alia, in Ng, Science 283 (1999), 2085-2089 or Ubarretxena-Belandia, Biochem. 38 (1999), 7398-7405), TR-FRETs and biochemical assays as disclosed herein. Furthermore, commercial assays like “Amplified Luminescent Proximity Homogenous Assay™” (BioSignal Packard) may be employed. Further methods are well known in the art and, inter alia, described in Fernandez, Curr. Opin. Chem. Biol. 2 (1998), 597-603.

The method for identifying and/or obtaining interacting compounds may also be carried out by specific immunological and/or biochemical assays which are well known in the art and which comprise, e.g., homogenous and heterogeneous assays as described herein below. Said interaction assays employing read-out systems are well known in the art and comprise, inter alia, two-hybrid screenings (as, described, inter alia, in EP-0 963 376, WO 98/25947, WO 00/02911; GST-pull-down columns, co-precipitation assays from cell extracts as described, inter alia, in Kasus-Jacobi, Oncogene 19 (2000), 2052-2059, “interaction-trap” systems (as described, inter alia, in U.S. Pat. No. 6,004,746) expression cloning (e.g. lamda gt11), phage display (as described, inter alia, in U.S. Pat. No. 5,541,109), in vitro binding assays and the like. Further interaction assay methods and corresponding read out systems are, inter alia, described in U.S. Pat. No. 5,525,490, WO 99/51741, WO 00/17221, WO 00/14271 or WO 00/05410. Vidal and Legrain (Nucleic Acids Research 27 (1999), 919-929) describe, review and summarize further interaction assays known in the art which may be employed in accordance with the present invention.

Homogeneous (interaction) assays comprise assays wherein the binding partners remain in solution and comprise assays, e.g., agglutination assays. Heterogeneous assays comprise assays like, inter alia, immuno assays, e.g., Enzyme Linked Immunosorbent Assays (ELISA), Radioactive Immunoassays (RIA), Immuno Radiometric Assays (IRMA), Flow Injection Analysis (FIA), Flow Activated Cell Sorting (FACS), Chemiluminescent Immuno Assays (CLIA) or Electrogenerated Chemiluminescent (ECL) reporting.

The present invention further provides a screening method for identifying and/or obtaining a compound which is an inhibitor or an antagonist of a polypeptide as listed in Tables 2 to 6 whose expression is up-regulated in HCV-infected tissue comprising the steps of a) contacting said polypeptide with a compound or a plurality of compounds under conditions which allow interaction of said compound with said polypeptide; b) determining the activity of said polypeptide; c) comparing the activity of said polypeptide in the presence of the compound or the plurality of compounds to the activity determined in the absence of the compound or the plurality of compounds; and identifying and/or obtaining an inhibitor or an antagonist, wherein a decreased activity determined in (c) is indicative for an inhibitor or antagonist. The terms inhibitors and antagonists as used herein are used interchangeably. The screening method can be performed either as an in vitro assay, or as a host-based assay. The host to be employed in the screening methods of the present invention and comprising and/or expressing a polypeptide listed as in Tables 2 to 6 may comprise prokaryotic as well as eukaryotic cells. Said cells may comprise bacterial cells, yeast cells, as well as cultured (tissue) cell lines, inter alia, derived from mammals. Furthermore animals may also be employed as hosts, for example non-human transgenic animals. Accordingly, said host (cell) may be transfected or transformed with the vector comprising a nucleic acid molecule encoding a polypeptide which is differentially expressed in HCV-infected tissue as disclosed herein. Said host cell or host may therefore be genetically modified with a nucleic acid molecule encoding such a polypeptide or with a vector comprising such a nucleic acid molecule. The present invention further provides a screening method for identifying and/or obtaining a compound which is an activator or agonist of a polypeptide as listed in Tables 1, 3 to 6 whose expression is down-regulated in HCV-infected tissue comprising the steps of a) contacting said polypeptide with a compound or a plurality of compounds under conditions which allow interaction of said compound with said polypeptide; b) determining the activity of said polypeptide; c) comparing the activity of said polypeptide in the presence of the compound or the plurality of compounds to the activity determined in the absence of the compound or the plurality of compounds; and identifying and/or obtaining an activator or agonist, wherein an increased activity determined in (c) is indicative for an activator or agonist. The terms activator and agonist as used herein are used interchangeably. The screening method can be performed either as an in vitro assay, or as a host-based assay. The host to be employed in the screening methods of the present invention and comprising and/or expressing a polypeptide listed as in Tables 1, 3 to 6 may comprise prokaryotic as well as eukaryotic cells. Said cells may comprise bacterial cells, yeast cells, as well as cultured (tissue) cell lines, inter alia, derived from mammals. Furthermore animals may also be employed as hosts, for example non-human transgenic animals. Accordingly, said host (cell) may be transfected or transformed with the vector comprising a nucleic acid molecule encoding a polypeptide which is differentially expressed in HCV-infected tissue as disclosed herein. Said host cell or host may therefore be genetically modified with a nucleic acid molecule encoding such a polypeptide or with a vector comprising such a nucleic acid molecule.

The term “genetically modified” means that the host cell or host comprises in addition to its natural genome a nucleic acid molecule or vector coding for a polypeptide as listed in Tables 2 to 6 or at least a fragment thereof. Said additional genetic material may be introduced into the host (cell) or into one of its predecessors/parents. The nucleic acid molecule or vector may be present in the genetically modified host cell or host either as an independent molecule outside the genome, preferably as a molecule which is capable of replication, or it may be stably integrated into the genome of the host cell or host.

As mentioned herein above, the host cell of the present invention may be any prokaryotic or eukaryotic cell. Suitable prokaryotic cells are those generally used for cloning like E. coli or Bacillus subtilis. Yet, these prokaryotic host cells are also envisaged in the screening methods disclosed herein. Furthermore, eukaryotic cells comprise, for example, fungal or animal cells. Examples for suitable fungal cells are yeast cells, preferably those of the genus Saccharomyces and most preferably those of the species Saccharomyces cerevisiae. Suitable animal cells are, for instance, insect cells, vertebrate cells, preferably mammalian cells, such as e.g. CHO, HeLa, NIH3T3 or MOLT-4. Further suitable cell lines known in the art are obtainable from cell line depositories, like the American Type Culture Collection (ATCC).

The hosts may also be selected from non-human mammals, most preferably mice, rats, sheep, calves, dogs, monkeys or apes. As described herein above, said animals/mammals also comprise non-human transgenic animals, which preferably express at least one polypeptide differentially expressed in HCV-infected cells or tissue as disclosed herein. Preferably, said polypeptide is a polypeptide, which is up-regulated in tissue derived from HCV-infected subjects. Yet it is also envisaged that non-human transgenic animals be produced which do not express the polypeptides as disclosed herein or which only express limited amounts of said polypeptides. Said animals are preferably related to polypeptides, which are down-regulated in HCV-infected tissue. Transgenic non-human animals comprising and/or expressing the up-regulated polypeptides of the present invention or alternatively, which comprise silenced or less efficient versions of down-regulated polypeptides are useful models for studying the course and/or outcome of a HCV infection and provide for useful models for testing drugs and therapeutics for treatment of HCV infection.

A compound which inhibits or antagonizes or which activates or agonizes a polypeptide as listed in Tables 1 to 6 is identified by determining the activity of said polypeptide in the presence of said compound.

The term “activity” as used herein relates to the functional property or properties of a specific polypeptide. For the polypeptides as listed in Tables 1 to 6 know as enzymes, the term “activity” relates to the enzymatic activity of the specific polypeptide. Activity assays for the enzymes as listed in Tables 1 to 6 are well known in the art.

For the polypeptides as listed in Tables 1 to 6 know as adhesion molecules, the term “activity” relates to the adhesive properties of a polypeptide and may be determined using assays such as, but not limited to, adhesion assays, cell spreading assays, or in vitro interaction assays of the adhesion molecule with a known ligand. Such assays are well known in the art.

For the polypeptides as listed in Tables 1 to 6 known as cytoskeletal proteins, the term “activity” relates to the regulation of the cytoskeleton by such polypeptides, or to their incorporation into the cytoskeleton. As a non-limiting example, the ability of Gelsolin to regulate actin polymerization, or of Filamin A to promote orthogonal branching of actin filaments, may be determined using in vitro actin polymerization assays. Activity in relation to the regulation of cytoskeletal structures may further be determined by, as non-limiting examples, cell spreading assays, cell migration assays, cell proliferation assays or immunofluorecence assays, or by staining actin filaments with fluorescently labeled phalloidin. All of these assays are well known to the person skilled in the art.

For the polypeptides as listed in Tables 1 to 6 known as ion channels the term “activity” relates to ion flux across the membrane. Methods to determine ion flux across membranes are well known to the person skilled in the art.

For polypeptides as listed in Tables 1 to 6 known as transcription factors, the term “activity” relates to their ability to regulate gene transcription. The transcriptional activity of a polypeptide can be determined using assays know in the art, such as a reporter gene assay.

For polypeptides as listed in Tables 1 to 6 known as growth factors and hormones or their receptors, the term “activity” relates to their ability to bind to their receptors or ligands, respectively, and to induce receptor activation and subsequent signaling cascades, and/or it relates to the ability of the factor or receptor to mediate the cellular function or functions eventually caused by growth factor or hormone mediated receptor activation. Growth factor or hormone binding to receptors can be determined by commonly known ligand binding assays. Receptor activation can be determined by testing for receptor autophosphorylation, or by assaying for modification or recruitment of downstream signaling mediators to the receptors (by immunoprecipitation and Western Blotting of signaling complexes). Cellular functions regulated by growth factors or hormones and their receptors can be cell proliferation (e.g. determined by using thymidine incorporation or cell counts), cell migration assays (e.g. determined by using modified Boyden chambers), cell survival or apoptosis assays (e.g. determined by using DAPI staining), angiogenesis assays (e.g. in vitro assays to measure endothelial tube formation that are commercially available). In addition to these assays, other assays may be used as well to determine these and other cellular functions.

Inhibitors or antagonists of a polypeptide as listed in Tables 2 to 6 are identified by the screening method described above when there is a decreased activity determined in the presence of the compound in comparison to the activity in the absence of the compound in the screening method.

Activators or agonists of a polypeptides as listed in Tables 1, 3 to 6 are identified by the described screening method of the invention when there is an increased activity determined in the presence of the compound as compared to the activity in the absence of the compound in the screening method.

Further to the screening methods disclosed above, this invention provides a screening method for identifying and/or obtaining a compound which is an inhibitor of the expression of a polypeptide selected from the polypeptides as to listed in Tables 2 to 6 whose expression is up-regulated in HCV-infected tissue, comprising the steps of a) contacting a host which expresses said polypeptide with a compound or a plurality of compounds; b) determining the expression level of said polypeptide; c) comparing the expression level of said polypeptide in the presence of the compound or the plurality of compounds to the expression level of said polypeptide determined in the absence of the compound or plurality of compounds; and d), identifying and/or obtaining an inhibitor of the expression of a polypeptide, wherein a decreased expression level determined in (c) is indicative for an inhibitor of the expression of said polypeptide.

Further to the screening methods disclosed above, this invention provides a screening method for identifying and/or obtaining a compound which is an activator of the expression of a polypeptide selected from the polypeptides as to listed in Tables 1, 3 to 6 whose expression is down-regulated in HCV-infected tissue, comprising the steps of a) contacting a host which expresses said polypeptide with a compound or a plurality of compounds; b) determining the expression level of said polypeptide; c) comparing the expression level of said polypeptide in the presence of the compound or the plurality of compounds to the expression level of said polypeptide determined in the absence of the compound or plurality of compounds; and d), identifying and/or obtaining an activator of the expression of a polypeptide, wherein a increased expression level determined in (c) is indicative for an activator of the expression of said polypeptide.

An inhibitor of the expression of a polypeptide as listed in Tables 2 to 6 is identified by the screening method described hereinbefore, when a decreased expression of the protein is determined in the presence of the compound in comparison to the absence of the compound in the screening method, which is indicative for an inhibitor of the expression of a polypeptide.

The term “expression” as used herein relates to amount determined of a polypeptide as listed in Tables 2 to 6 which is up-regulated or down-regulated in HCV-infected tissue, in HCV-infected cells, which is elevated or reduced as compared to the amount of the same polypeptide in uninfected tissue or uninfected cells. Preferably, expression levels are at least 2 fold, more preferably at least 3 fold, even more preferably at least 4 fold, most preferably at least 5 fold higher or lower than in uninfected tissue or cells.

Furthermore, the present invention provides a compound identified and/or obtained by any of the screening methods hereinbefore described. The compounds identified and/or obtained by any of the screening methods of the present invention comprise small molecules, polypeptides, hormones, polynucleotides comprising antisense nucleotides and antibodies. A pharmaceutical composition comprising said compound and pharmaceutically acceptable carrier or diluent is further provided. A method for the preparation of said pharmaceutical composition comprising formulating said compound in a pharmaceutically acceptable carrier or diluent is also claimed. Any conventional carrier material can be utilized. The carrier material can be an organic or inorganic one suitable for eteral, percutaneous or parenteral administration. Suitable carriers include water, gelatin, gum arabic, lactose, starch, magnesium stearate, talc, vegetable oils, polyalkylene-glycols, petroleum jelly and the like. Furthermore, the pharmaceutical preparations may contain other pharmaceutically active agents. Additional additives such as flavoring agents, stabilizers, emulsifying agents, buffers and the like may be added in accordance with accepted practices of pharmaceutical compounding.

Said compound may be used for the preparation of a medicament for the treatment or prevention of HCV infection. Preferably, said compound is selected from the group comprising an antibody, an antibody-derivative, an antibody fragment, a peptide or an antisense construct.

Within the scope of the present invention, antibodies against the polypeptides as listed in Tables 1 to 6, or antigen-binding fragments thereof, for the use in an in vitro method for the prediction of the outcome of a HCV infection in a subject are provided.

In order to efficiently perform the prognostic method, the present invention provides a kit for the prediction of the outcome of a HCV infection in a subject comprising one or more of the antibodies, or antigen-binding fragments thereof as described above. Another kit provided by the invention is a kit for screening of compounds that activate or inhibit the activity, or activate or inhibit the expression, of any one of the polypeptides as listed in Tables 1 and 2.

Having now generally described this invention, the same will become better understood by reference to the specific examples, which are included herein for purpose of illustration only and are not intended to be limiting unless otherwise specified.

TABLE 1

| | |
|---|---|
| SEQ ID NO | Down-regulated polypeptides |

| | |
|---|---|
| 1 | HSUGP:006101-3-0 |
| 2 | HUMANGP:CHR11-Q9UMS4 |
| 3 | HUMANGP:CHR17-Q8TCD5 |
| 4 | HUMANGP:CHR19-Q9BVL0 |
| 5 | HUMANGP:CHR1-CAC22475 |
| 6 | HUMANGP:CHR1-Q9HB71 |
| 7 | HUMANGP:CHR4-Q96S43 |
| 8 | HUMANGP:CHR4-Q9Z130 |
| 9 | HUMANGP:CHR7-Q13122 |
| 10 | HUMANGP:CHR9-O60376 |
| 11 | HUMANGP:CHR9-Q9BS26 |
| 12 | SW_HUM:ACK1_HUMAN |
| 13 | SW_HUM:ACK2_HUMAN |
| 14 | SW_HUM:ALFC_HUMAN |
| 15 | SW_HUM:APT_HUMAN |
| 16 | SW_HUM:ARG1_HUMAN |
| 17 | SW_HUM:B53A_HUMAN |
| 18 | SW_HUM:CATB_HUMAN |
| 19 | SW_HUM:CN03_HUMAN |
| 20 | SW_HUM:COF1_HUMAN |
| 21 | SW_HUM:COPE_HUMAN |
| 22 | SW_HUM:DEST_HUMAN |
| 23 | SW_HUM:DHA5_HUMAN |
| 24 | SW_HUM:DHQU_HUMAN |
| 25 | SW_HUM:DPP3_HUMAN |
| 26 | SW_HUM:ECH1_HUMAN |
| 27 | SW_HUM:EF1D_HUMAN |
| 28 | SW_HUM:ENOL_HUMAN |
| 29 | SW_HUM:FIBG_HUMAN |
| 30 | SW_HUM:G3B2_HUMAN |
| 31 | SW_HUM:G3BP_HUMAN |
| 32 | SW_HUM:G6PD_HUMAN |
| 33 | SW_HUM:GAMT_HUMAN |
| 34 | SW_HUM:GRF1_HUMAN |
| 35 | SW_HUM:GTO1_HUMAN |
| 36 | SW_HUM:H105_HUMAN |
| 37 | SW_HUM:HDGF_HUMAN |
| 38 | SW_HUM:HMCS_HUMAN |
| 39 | SW_HUM:IF2A_HUMAN |
| 40 | SW_HUM:1F33_HUMAN |
| 41 | SW_HUM:1F41_HUMAN |
| 42 | SW_HUM:LAM1_HUMAN |
| 43 | SW_HUM:LAM2_HUMAN |
| 44 | SW_HUM:LAMA_HUMAN |
| 45 | SW_HUM:LDHA_HUMAN |
| 46 | SW_HUM:MAE1_HUMAN |
| 47 | SW_HUM:MAOX_HUMAN |
| 48 | SW_HUM:MDHC_HUMAN |
| 49 | SW_HUM:NDKA_HUMAN |
| 50 | SW_HUM:NDR1_HUMAN |
| 51 | SW_HUM:PCNA_HUMAN |
| 52 | SW_HUM:PGK1_HUMAN |
| 53 | SW_HUM:PRS6_HUMAN |
| 54 | SW_HUM:PSB7_HUMAN |
| 55 | SW_HUM:R23B_HUMAN |
| 56 | SW_HUM:ROC_HUMAN |
| 57 | SW_HUM:ROH2_HUMAN |
| 58 | SW_HUM:ROH3_HUMAN |
| 59 | SW_HUM:SC13_HUMAN |
| 60 | SW_HUM:SNAA_HUMAN |
| 61 | SW_HUM:SPEE_HUMAN |
| 62 | SW_HUM:TKT_HUMAN |
| 63 | SW_HUM:UBA1_HUMAN |
| 64 | SW_HUM:UBC1_HUMAN |
| 65 | SW_HUM:UBPE_HUMAN |
| 66 | SW_HUM:UNRI_HUMAN |
| 67 | SW_HUM:VAA1_HUMAN |
| 68 | SW_HUM:VIL1_HUMAN |
| 69 | TR_HUM:Q96HX0 |

TABLE 2

| | |
|---|---|
| SEQ ID NO | Up-regulated polypeptides |

| | |
|---|---|
| 70 | SW_HUM:PTB_HUMAN |
| 71 | SW_HUM:KPY2_HUMAN |
| 72 | SW_HUM:ACDB_HUMAN |
| 73 | SW_HUM:RS4_HUMAN |
| 74 | SW_HUM:CRKL_HUMAN |
| 75 | SW_HUM:DDX5_HUMAN |
| 76 | SW_HUM:LAS1_HUMAN |
| 77 | SW_HUM:SYY_HUMAN |
| 78 | SW_HUM:SEP7_HUMAN |
| 79 | SW_HUM:ATPO_HUMAN |
| 80 | SW_HUM:MCM3_HUMAN |
| 81 | SW_HUM:RL12_HUMAN |
| 82 | TR_HUM:Q9Y280 |
| 83 | SW_HUM:AMRP_HUMAN |
| 84 | SW_HUM:ARG2_HUMAN |
| 85 | SW_HUM:IF2B_HUMAN |
| 86 | SW_HUM:ROG_HUMAN |
| 87 | HUMANGP:CHR11-O75932 |
| 88 | SW_HUM:TCPD_HUMAN |
| 89 | HSUGP:075546-8-0 |
| 90 | HUMANGP:CHR3-O75191 |
| 91 | SW_HUM:DUS9_HUMAN |
| 92 | SW_HUM:GTM4_HUMAN |
| 93 | SW_HUM:RS8_HUMAN |
| 94 | SW_HUM:SM30_HUMAN |
| 95 | HSUGP:013561-2-0 |
| 96 | HUMANGP:CHR2-Q9UQE6 |
| 97 | HUMANGP:CHR7-Q9UNH0 |
| 98 | SW_HUM:PEBB_HUMAN |
| 99 | HSUGP:075309-23-0 |
| 100 | HSUGP:075309-24-0 |
| 101 | SW_HUM:NR54_HUMAN |
| 102 | SW_HUM:DDXY_HUMAN |
| 103 | HUMANGP:CHR8-O94905 |
| 104 | SW_HUM:COPD_HUMAN |
| 105 | SW_HUM:CAPB_HUMAN |
| 106 | SW_HUM:VATE_HUMAN |
| 107 | SW_HUM:ACLY_HUMAN |
| 108 | HSUGP:008768-9-0 |
| 109 | HSUGP:075546-6-0 |
| 110 | SW_HUM:DD17_HUMAN |
| 111 | SW_HUM:CAPG_HUMAN |
| 112 | SW_HUM:VAB1_HUMAN |
| 113 | SW_HUM:UBP5_HUMAN |
| 114 | TR_HUM:Q9BVH9 |
| 115 | SW_HUM:CGL1_HUMAN |
| 116 | SW_HUM:VIME_HUMAN |
| 117 | SW_HUM:PRO1_HUMAN |
| 118 | SW_HUM:RS2_HUMAN |
| 119 | SW_HUM:NADC_HUMAN |
| 120 | HUMANGP:CHR5-Q96NM0 |
| 121 | HUMANGP:CHR19-O95717 |
| 122 | SW_HUM:GNPI_HUMAN |
| 123 | SW_HUM:NAGK_HUMAN |
| 124 | SW_HUM:SODM_HUMAN |
| 125 | SW_HUM:ESTD_HUMAN |
| 126 | SW_HUM:GLYM_HUMAN |
| 127 | SW_HUM:IDHP_HUMAN |
| 128 | SW_HUM:PSA1_HUMAN |
| 129 | SW_HUM:K6A3_HUMAN |
| 130 | HUMANGP:CHR20-O14878 |
| 131 | SW_HUM:PAK2_HUMAN |
| 132 | SW_HUM:MEPD_HUMAN |
| 133 | SW_HUM:PSB5_HUMAN |
| 134 | SW_HUM:MK01_HUMAN |
| 135 | SW_HUM:MCCB_HUMAN |
| 136 | SW_HUM:RRB1_HUMAN |
| 137 | TR_HUM:Q96E67 |
| 138 | SW_HUM:HGD_HUMAN |
| 139 | SW_HUM:6PGD_HUMAN |
| 140 | SW_HUM:IF4N_HUMAN |
| 141 | SW_HUM:UCR2_HUMAN |
| 142 | SW_HUM:143Z_HUMAN |
| 143 | SW_HUM:TAL1_HUMAN |
| 144 | SW_HUM:NPS1_HUMAN |
| 145 | SW_HUM:DCUP_HUMAN |
| 146 | SW_HUM:AATM_HUMAN |
| 147 | SW_HUM:PSA2_HUMAN |
| 148 | SW_HUM:KPY1_HUMAN |
| 149 | SW_HUM:AATC_HUMAN |
| 150 | SW_HUM:ATPA_HUMAN |
| 151 | SW_HUM:E2BG_HUMAN |
| 152 | SW_HUM:GLPK_HUMAN |
| 153 | SW_HUM:RS2_HUMAN |
| 154 | SW_HUM:SYFA_HUMAN |
| 155 | SW_HUM:DJA1_HUMAN |
| 156 | SW_HUM:SYSM_HUMAN |
| 157 | SW_HUM:HAXI_HUMAN |
| 158 | HUMANGP:CHR19-Q96G16 |
| 159 | HUMANGP:CHR10-Q9HBK9 |
| 160 | SW_HUM:SPS1_HUMAN |
| 161 | SW_HUM:IF39_HUMAN |
| 162 | SW_HUM:NCB1_HUMAN |
| 163 | SW_HUM:Y33K_HUMAN |
| 164 | SW_HUM:HEM6_HUMAN |
| 165 | SW_HUM:THIM_HUMAN |
| 166 | SW_HUM:SRC8_HUMAN |
| 167 | SW_HUM:KAD4_HUMAN |
| 168 | HUMANGP:CHR12-Q9NRV9 |
| 169 | HUMANGP:CHR12-Q14221 |
| 170 | SW_HUM:MYG1_HUMAN |
| 171 | SW_HUM:MPPA_HUMAN |
| 172 | SW_HUM:PSDB_HUMAN |
| 173 | SW_HUM:ACDM_HUMAN |
| 174 | SW_HUM:GIPC_HUMAN |
| 175 | HUMANGP:CHR1-AAH15403 |
| 176 | SW_HUM:GATM_HUMAN |
| 177 | SW_HUM:PSE3_HUMAN |
| 178 | SW_HUM:CALU_HUMAN |
| 179 | SW_HUM:DCT2_HUMAN |
| 180 | SW_HUM:HCD2_HUMAN |
| 181 | SW_HUM:PDXK_HUMAN |
| 182 | SW_HUM:IF6_HUMAN |
| 183 | SW_HUM:PDX5_HUMAN |
| 184 | SW_HUM:PP1G_HUMAN |
| 185 | SW_HUM:PIMT_HUMAN |
| 186 | SW_HUM:TCPQ_HUMAN |
| 187 | HSUGP:333417-11-0 |
| 188 | SW_HUM:PSB3_HUMAN |
| 189 | HUMANGP:CHR6-Q8TDM4 |
| 190 | SW_HUM:BIEA_HUMAN |
| 191 | SW_HUM:RAN_HUMAN |
| 192 | SW_HUM:PRS8_HUMAN |
| 193 | HUMANGP:CHR1-O14805 |
| 194 | SW_HUM:PSA6_HUMAN |
| 195 | SW_HUM:6PGL_HUMAN |
| 196 | HUMANGP:CHR19-Q8WVX0 |
| 197 | SW_HUM:ACON_HUMAN |
| 198 | SW_HUM:UCR1_HUMAN |
| 199 | SW_HUM:HS27_HUMAN |

TABLE 3

| | Up- or downregulated m_RNA |
|---|---|
| | binding poteins and mRNA |
| SEQ ID NO | chaperones (selected from Tables 1 and 2) |
| 70 | PTB_HUMAN |
| 101 | NR54_HUMAN |
| 75 | DDX5_HUMAN |
| 110 | DD17_HUMAN |
| 102 | DDXY_HUMAN |
| 86 | ROG_HUMAN |
| 82 | Q9Y280 |

TABLE 4

| | Up- or downregulated proteins |
|---|---|
| | involved in the regulation of protein |
| SEQ ID NO | synthesis (selected from Tables 1 and 2) |
| 85 | IF2B_HUMAN |
| 151 | E2BG_HUMAN |
| 161 | IF39_HUMAN |
| 140 | IF4N_HUMAN |
| 182 | IF6_HUMAN |
| 136 | RRB1_HUMAN |
| 129 | K6A3_HUMAN |

TABLE 5

| | Up- or downregulated proteins |
|---|---|
| | involved in lipid metabolism |
| SEQ ID NO | (selected from Tables 1 and 2) |
| 180 | HDC2_HUMAN |
| 173 | ACDM_HUMAN |
| 72 | ACDB_HUMAN |
| 135 | MCCB_HUMAN |
| 165 | THIM_HUMAN |

TABLE 6

| | Up- or downregulated proteins |
|---|---|
| | involved in signal transduction |
| SEQ ID NO | pathways (selected from Tables 1 and 2) |
| 91 | DUS9_HUMAN |
| 98 | PEBB_HUMAN |
| 166 | SCR8_HUMAN |
| 157 | HAX1_HUMAN |
| 131 | PAK2_HUMAN |
| 193 | O14805 |

### EXAMPLES

Commercially available reagents referred to in the examples were used according to manufacturer's instructions unless otherwise indicated.

### Example 1

Huh 8.1 cell line was grown under standard conditions (control) and compared to Huh 9.13 (replicon). The Huh 9.13 cell line was prepared according to Lohmann, V. et al., Science 1999, 285, 110-113. The growth experiment was repeated 3 times, different cell lysis conditions were employed each time (Lohmann, V, et al., (1999), Science 285, 110-113; Bartenschläger, R. (2002), Nature Reviews, Hepatitis C virus replicons: potential role for drug development, Vol. 1, 911-916).

Several standard cell fractions using differential centrifugation (800 g, 12000 g, 60000 g) were generated. In addition, a particle fraction enriched in replicon protein (replicon particle) was isolated by differential centrifugation/density gradient centrifugation. For all fractions, sample/control pairs of 2D PAGE were run under identical conditions using IPG ranges from 3-10 and 4-7/6-10, and all accessible protein spots were identified using MALDI MS peptide fingerprinting.

[image]

For comparison, the results of all experiments were combined and counts of protein identifications were generated. A protein was assigned as “up-regulated” (“down-regulated”) if it was found in at least two samples and if it was either totally absent in the control (respective replicon sample) or the quotient between the count of mass spectra identified as the protein in the sample and control was larger than 2 (smaller than 0.5).

Polypeptides down-regulated by the presence of HCV replicon-derived RNA are depicted in Table 1, and polypeptides up-regulated by the presence of HCV replicon derived RNA are depicted in Table 2, comprising a number of interesting mRNA binding proteins and dysregulated components of the protein synthesis machinery.

1. An in vitro method for the prediction of the outcome of a HCV infection in a subject comprising obtaining a biological sample from a HCV-infected subject and determining the expression level of at least one polypeptide selected from SEQUENCE ID NOS. 1-199.

2. The method of claim 1, further comprising comparing the expression level of the at least one polypeptide to its expression level determined at another time point in the course of a HCV infection or to its expression level in a biological sample from a subject uninfected with HCV.

3. The method of claim 1 wherein the polypeptide is selected from the group consisting of SEQUENCE ID NOS. 70, 75, 82, 86, 101, 102 and 110.

4. The method of claim 1 wherein the polypeptide is selected from the group consisting of SEQUENCE ID NOS. 85, 129, 136, 140, 151, 161, and 182.

5. The method of claim 1 wherein the polypeptide is selected from the group consisting of SEQUENCE ID NOS. 72, 135, 165, 173, and 180.

6. The method of claim 1 wherein the polypeptide is selected from the group consisting of SEQUENCE ID NOS. 91, 98, 131, 157, 166 and 193.

7. A screening method for identifying a compound which interacts with at least one polypeptide selected from the polypeptides of SEQUENCE ID NOS. 1-199 whose expression is up-regulated or down-regulated in HCV-infected tissue comprising the steps of
a) contacting said polypeptide with a compound or a plurality of compounds under conditions which allow interaction of said compound with said polypeptide; and
b) determining whether the activity of said polypeptide is modified.

8. A screening method for identifying a compound which is an inhibitor or an antagonist of a polypeptide of SEQUENCE ID NOS. 70-199 whose expression is up-regulated in HCV-infected tissue, comprising the steps of
a) contacting said polypeptide with a compound or a plurality of compounds under conditions which allow interaction of said compound(s) with said polypeptide;
b) determining the activity of said polypeptide;
c) comparing the activity of said polypeptide in the presence of the compound(s) to the activity determined in the absence of the compound(s);
wherein a decreased activity determined in (c) is indicative of inhibitor or antagonist activity of such compound(s).

9. A screening method for identifying a compound which is an activator or an agonist of a polypeptide of SEQUENCE ID NOS. 1-69 whose expression is down-regulated in HCV-infected tissue, comprising the steps of
a) contacting said polypeptide with a compound or a plurality of compounds under conditions which allow interaction of said compound(s) with said polypeptide;
b) determining the activity of said polypeptide;
c) comparing the activity of said polypeptide in the presence of the compound(s) to the activity determined in the absence of the compound(s);
wherein an increased activity determined in (c) is indicative of activator or agonist activity of such compound(s).

10. The method of claim 8 wherein the polypeptide is selected from the group consisting of SEQUENCE ID NOS. 70, 75, 82, 86, 101, 102 and 110.

11. The method of claim 8 wherein the polypeptide is selected from the group consisting of SEQUENCE ID NOS. 85, 129, 136, 140, 151, 161, and 182.

12. The method of claim 8 wherein the polypeptide is selected from the group consisting of SEQUENCE ID NOS. 72, 135, 165, 173, and 180.

13. The method of claim 8 wherein the polypeptide is selected from the group consisting of SEQUENCE ID NOS. 91, 98, 131, 157, 166 and 193.

14. A screening method for identifying a compound which is an inhibitor of the expression of a polypeptide selected from the polypeptides of SEQUENCE ID NOS. 70-199 whose expression is up-regulated in HCV-infected tissue, comprising the steps of
a) contacting a host cell which expresses said polypeptide with a compound or a plurality of compounds;
b) determining the expression level of said polypeptide;
c) comparing the expression level determined in the presence of the compound(s) to the expression level determined in the absence of the compound(s);
wherein a decreased expression level determined in (c) is indicative of an inhibitor of the expression of said polypeptide.

15. The method of claim 14 wherein the polypeptide is selected from the group consisting of SEQUENCE ID NOS. 70, 75, 82, 86, 101, 102 and 110.

16. The method of claim 14 wherein the polypeptide is selected from the group consisting of SEQUENCE ID NOS. 85, 129, 136, 140, 151, 161, and 182.

17. The method of claim 14 wherein the polypeptide is selected from the group consisting of SEQUENCE ID NOS. 72, 135, 165, 173, and 180.

18. The method of claim 14 wherein the polypeptide is selected from the group consisting of SEQUENCE ID NOS. 91, 98, 131, 157, 166 and 193.

19. A screening method for identifying a compound which is an activator of the expression of a polypeptide selected from the polypeptides of SEQUENCE ID NOS. 1-69 whose expression is down-regulated in HCV-infected tissue, comprising the steps of
a) contacting a host cellwhich expresses said polypeptide with a compound or a plurality of compounds;
b) determining the expression level of said polypeptide;
c) comparing the expression level determined in the presence of the compound(s) to the expression level determined in the absence of the compound(s);
wherein an increased expression level determined in (c) is indicative of an activator of the expression of said polypeptide.

20. A compound identified by the screening method of claim 14.

21. A compound identified by the screening method of claim 19.

22. A pharmaceutical composition comprising the compound of claim 20 and a pharmaceutically acceptable carrier or diluent.

23. A pharmaceutical composition comprising the compound of claim 21 and a pharmaceutically acceptable carrier or diluent.

24. A method for treating HCV infection comprising administering a pharmaceutically effective amount of the composition of claim 17 to a patient infected with HCV.

25. A method for treating HCV infection comprising administering a pharmaceutically effective amount of the composition of claim 18 to a patient infected with HCV.

26. An antibody composition comprising an antibody or the antigen-binding fragment of an antibody that selectively binds to a polypeptide selected from the group consisting of SEQUENCE ID NOS. 1-199

27. A kit for the prediction of the outcome of a HCV infection in a subject comprising one or more of the antibody compositions, of claim 26.

28. A target host cell for testing an antiviral compound comprising a cell which is genetically modified so as to express at least one polypeptide selected from SEQUENCE ID NOS. 1-199.
